(19) United States
(12) Patent Application Publication
(10) Pub. No.: US 2007/0167362 A1
(43) Pub. Date: Jul. 19, 2007

(54) Medicines containing nerve growth factor for assisting losing weight and methods for assisting losing weight using same

(75) Inventors: ZhiWen Zhou, Beijing (CN); YuXia Feng, Beijing (CN); Conglin Zuo, Beijing (CN)

Correspondence Address: SUGHRUE MION, PLLC, 2100 PENNSYLVANIA AVENUE, N.W., SUITE 800, WASHINGTON, DC, 20037, US

(73) Assignee: Staidson (Beijing) Pharmaceutical Co., Ltd.

(21) Appl. No.: 11/333,348

(22) Filed: Jan. 18, 2006

(51) Int. Cl. A61K 38/18

(52) U.S. Cl. 514/12

(57) ABSTRACT

A medicine for assisting losing weight contains nerve growth factor. Further, a method for assisting losing weight includes administering an effective amount of this medicine for assisting losing weight to a subject in need thereof.

### TECHNICAL FIELD

This invention relates to the application of nerve growth factor (NGF) in the preparation of medicines that can effectively assist losing weight. This invention belongs in the biomedical field.

### BACKGROUND

Weight is mainly determined by the relative balance between food intake and energy consumption. Although some other factors such as inheritance may result in obesity, the most common causes for weight gain or physical obesity are too much high-calorie food intake and/or decreasing of energy consumption. For overweight or obese people, excessive fat increases the incidence of cardiocerebrovascular or respiratory diseases, including hypertension, hyperlipemia, non-insulin dependent diabetes mellitus, coronary heart disease, sleep apnea syndrome and so on.

Obesity can be reduced or prevented through appropriate diet, lifestyle change or medication therapy. Common medications include orlistat (which can decrease the amount of fat absorbed by the intestine tract from the food), sibutramine (which can restrain appetite by inhibiting the reuptake of norepinephrine and 5-hydroxytryptamine), and fenfluramine (which can restrain appetite by releasing 5-hydroxytryptamine and subsequently inhibiting its reuptake). However, the majority of the common medications cause slight or severe adverse reactions. For instance, fenfluramine was clinically proven to significantly increase the risk of cardiovascular damage. For this reason, fenfluramine was removed from the market by the FDA in 1999. Other common adverse effects of these drugs include dizziness, headache, palpitation, insomnia, diarrhea, intestinal tract spasm, hypertension and so on. In addition to these adverse effects, many drugs such as amphetamine are addictive.

Nerve growth factor (NGF) is a neurocyte regulatory factor. It was discovered in the tumor cell of a rat by Rita Levi-Montalcini, an Italy embryologist, and Stanley Cohen, an American chemist, in 1953. NGF is mainly from targeted cells dominated by neural crest neuron. For example, there are rich NGFs in the central gial cells, peripheral Schwann's cells, skeletal muscles and other glands, the submaxillary gland of adult rats, bovine semen, venom, and human placentas.

Over the past more than fifty years, the research on NGF has become more extensive, and has shown that NGF has remarkable effects on the trauma of nerves, the immune system and other systems, but the adverse effects of NGF on animals or humans have not been found.

Nowadays, the drugs on the market whose main ingredient is NGF, are mostly used to treat the diseases such as damage to hand and foot nerves, Alzheimer's disease, nervous tissue transplantation, cerebral ischemia, peripheral neuritis and so on. However, currently, there have been no reports relating to the treatment of obesity with NGF.

### SUMMARY OF THE INVENTION

One objective of this invention is to provide a medicine for assisting losing weight effectively. Another objective of this invention is to provide a method for assisting losing weight.

These objects of the invention and others are achieved by the following:
(1) A medicine for assisting losing weight comprising NGF;
(2) The medicine described in item (1) above, which is an injection preparation or a nasal preparation.
(3) The medicine described in item (2) above, wherein the injection preparation is a solution injection, a suspension injection, an emulsion injection, a lyophilized powder for injection or an aseptic powder for injection.
(4) The medicine described in item (2) above, wherein the nasal preparation is nasal spray, a nasal drop, a nasal solution, a nasal pack or a nasal aerosol.
(5) A method for assisting losing weight comprising administering an effective amount of a medicine for assisting losing weight to a subject in need thereof, wherein the medicine comprises nerve growth factor.
(6) The method for assisting losing weight described in item (5) above, wherein the medicine is an injection preparation or a nasal preparation.
(7) The method for assisting losing weight described in item (6) above, wherein the injection preparation is a solution injection, a suspension injection, an emulsion injection, a lyophilized powder for injection or an aseptic powder for injection.
(8) The method for assisting losing weight described in item (6) above, wherein the nasal preparation is nasal spray, a nasal drop, a nasal solution, a nasal pack or a nasal aerosol.
(9) The method for assisting losing weight described in item (5) above, wherein the subject is a mammal.

### DETAILED DESCRIPTION OF THE INVENTION

The present invention is now described in detail in the following non-limiting technical protocols:

NGF is applied in the preparation of drugs that can assist losing weight effectively.

Our studies on NGF showed that the rats had minimal weight gain two weeks after administration of NGF, and the effect was dose-dependent. More than four weeks after administration, this phenomena persisted.

Compared to the drugs currently available on the market for assisting losing weight, the present invention can effectively assist losing weight. NGF can directly control weight gain. Further, weight gain doesn't occur after the administration of NGF has been terminated. In addition, the present invention does not cause any adverse reactions. In particular, NGF has no obvious adverse effects and is not addictive. Consequently, NGF can be used to effectively decrease weight.

### EXAMPLES

While the present invention will be more specifically described below in conjunction with Examples, it is not limited thereto.

### Example 1

This experiment confirmed the phenomena that NGF can restrain weight gain.

In this example, an injection or nasal spray of NGF was given to a rat and then the weight changes of the rat were monitored.

### 1. Drug

### Nerve growth factor (NGF) injection, and nasal spray of NGF were supplied by Beijing Joinn Pharmaceutical Center.

### 2. Animals

### 40 Wister rats (bought from the Laboratory Animal Center of Military Medical Academy), all male, were fed with standard beverage.

These rats were administrated an injection or nasal spray of NGF, respectively, at a dose of 4000 unit/kg. The blank control group was the rats received normal breeding. The weight of each rat was measured at the time of the administration (W0), and 2 weeks (W2), 4 weeks (W4), 6 weeks (W6), 8 weeks (W8), 10 weeks (W10), 12 weeks (W12), and 15 weeks (W15) following the administration, respectively.

The results are summarized in following Tables 1 and 2:

TABLE 1
The impact of NGF on the weight of male rats

| The | | | |
|---|---|---|---|
| number of | Weight (x ± SD, gram) | | |
| weeks in | | | Nasal Cavity |
| the | | Intramuscular Injection | Administration |
| experiment | Control Group | Group | Group |
| W0 | 179.8 ± 13.7 | 170.0 ± 8.9 | 174.0 ± 13.3 |
| W2 | 282.5 ± 23.6 | 255.9 ± 21.4** | 226.3 ± 17.1** |
| W4 | 339.9 ± 28.8 | 312.0 ± 29.9** | 270.5 ± 23.8** |
| W6 | 392.0 ± 36.4 | 358.0 ± 39.1** | 334.5 ± 32.0** |
| W8 | 422.3 ± 39.8 | 389.8 ± 44.5* | 365.1 ± 40.3** |
| W10 | 458.2 ± 42.0 | 422.6 ± 46.9* | 398.8 ± 47.6** |
| W12 | 454.5 ± 43.7 | 413.3 ± 45.2 | 407.0 ± 46.6** |
| W15^{Δ} | 505.00 ± 58.3 | 463.00 ± 81.0 | 444.00 ± 36.3 |

n = 20,
*p < 0.05,
**p < 0.01, compared with the control group during the corresponding period, there is statistically significant difference,
^{Δ}stands for restoration stage,
n = 5.

TABLE 2
The impact of NGF on weight increase of male rats

| The | | | |
|---|---|---|---|
| number of | Weight (x ± SD, gram/day/rat) | | |
| weeks in | | | Nasal Cavity |
| the | | Intramuscular Injection | Administration |
| experiment | Control Group | Group | Group |
| W0~W2 | 6.16 ± 1.49 | 5.06 ± 1.25* | 3.08 ± 0.79* |
| W2~W4 | 4.52 ± 1.D95 | 4.32 ± 1.29 | 3.40 ± 1.74 |
| W4~W6 | 2.90 ± 1.15 | 2.56 ± 1.20 | 3.56 ± 1.39 |
| W6~W8 | 2.02 ± 2.04 | 2.12 ± 0.62 | 2.37 ± 0.68 |
| W8~W10 | 2.22 ± 0.82 | 2.05 ± 0.98 | 1.73 ± 1.41 |
| W10~W12 | −0.61 ± 5.32 | −0.68 ± 1.82 | 1.15 ± 2.77 |
| W12~W15^{Δ} | 1.41 ± 0.46 | 1.51 ± 0.76 | 2.06 ± 0.66 |

n = 20,
*p < 0.05,
**p < 0.01_{W0~W4} Compared with the control group during the corresponding period, there is statistically significant difference,
^{Δ}stands for restoration stage,
n = 5.

### Example 2

This experiment confirmed the phenomena that NGF can restrain weight gain.

An injection or nasal spray of NGF was given to a rat and then the weight changes of the rat were monitored.

### Materials:

### 1. Drug

### Nerve growth factor (NGF) injection and NGF nasal spray were supplied by Beijing Joinn Pharmaceutical Center.

### 2. Animals

### 40 Wister rats (bought from the Laboratory Animal Center of Academy of Military Medical Sciences), all female, were fed with standard beverage.

The rats were administrated an injection or nasal spray of NGF, respectively, at a dose of 4000 unit/kg. The blank control group was the rat received the normal breeding. The weight of each rat was measured at the time of the administration (W0), and 2 weeks (W2), 4 weeks (W4), 6 weeks (W6), 8 weeks (W8), 10 weeks (W10), 12 weeks (W12), and 15 weeks (W15) following the administration, respectively.

The results are summarized in following Tables 3 and 4:

TABLE 3
The impact of NGF on the weight of female rats

| The | | | |
|---|---|---|---|
| number of | Weight (x ± SD, gram) | | |
| weeks in | | | Nasal Cavity |
| the | | Intramuscular Injection | Administration |
| experiment | Control Group | Group | Group |
| W0 | 170.2 ± 10.4 | 171.5 ± 11.2 | 163.9 ± 11.4 |
| W2 | 227.2 ± 11.0 | 226.8 ± 17.1 | 203.0 ± 14.2** |
| W4 | 256.5 ± 10.9 | 256.8 ± 20.8 | 234.0 ± 15.2** |
| W6 | 276.5 ± 17.9 | 288.0 ± 30.9 | 265.0 ± 23.3 |
| W8 | 290.1 ± 26.3 | 295.4 ± 29.1 | 278.5 ± 26.5 |
| W10 | 295.1 ± 21.6 | 305.5 ± 30.2 | 293.2 ± 30.7 |
| W12 | 306.2 ± 25.6 | 317.8 ± 30.1 | 300.6 ± 27.3 |
| W15^{Δ} | 321.00 ± 39.3 | 322.0 ± 32.9 | 316.00 ± 36.6 |

n = 20,
*p < 0.05,
**p < 0.01_{W2} Compared with the control group during the corresponding period, there is statistically significant difference,
^{Δ}stands for restoration stage,
n = 5.

TABLE 4
The impact of NGF on weight increase of female rats

| The | | | |
|---|---|---|---|
| number of | Weight (x ± SD, gram/day/rat) | | |
| weeks in | | | Nasal Cavity |
| the | | Intramuscular Injection | Administration |
| experiment | Control Group | Group | Group |
| W0~W2 | 3.45 ± 0.93 | 3.32 ± 0.67 | 2.30 ± 0.84** |
| W2~W4 | 2.25 ± 0.91 | 2.39 ± 1.12 | 2.39 ± 0.80 |
| W4~W6 | 1.14 ± 0.71 | 1.62 ± 1.56 | 1.72 ± 0.72* |
| W6~W8 | 0.83 ± 0.97 | 0.50 ± 1.81 | 1.03 ± 0.62 |
| W8~W10 | 0.33 ± 0.83 | 0.32 ± 1.11 | 0.80 ± 0.54* |
| W10~W12 | 1.10 ± 1.02 | 1.19 ± 1.10 | 0.89 ± 1.49 |
| W12~W15^{Δ} | 0.78 ± 0.38 | 0.86 ± 0.43 | 0.92 ± 1.05 |

n = 20,
*p < 0.05,
**p < 0.01_{W0~W2} Compared with the control group during the corresponding period, there is statistically significant difference,
^{Δ}stands for restoration stage,
n = 5.

The injection or nasal spray of NGF according to this invention can prevent weight gain, and the effect is dose-dependent. No adverse effects have been observed during the experiments or the restoration stages. Consequently, NGF would be a new drug that can assist losing weight effectively without any adverse effects. Those skilled in the art will appreciate any changes which can be made without departing from the scope and spirit of the present disclosure.

What is claimed is:

1. A medicine for assisting losing weight comprising nerve growth factor.

2. The medicine for assisting losing weight in claim 1, wherein the medicine is an injection preparation or a nasal preparation.

3. The medicine for assisting losing weight in claim 2, wherein the injection preparation is a solution injection, a suspension injection, an emulsion injection, a lyophilized powder for injection or an aseptic powder for injection.

4. The medicine for assisting losing weight in claim 2, wherein the nasal preparation is nasal spray, a nasal drop, a nasal solution, a nasal pack or a nasal aerosol.

5. A method for assisting losing weight comprising administering an effective amount of a medicine for assisting losing weight to a subject in need thereof, wherein the medicine comprises nerve growth factor.

6. The method for assisting losing weight in claim 5, wherein the medicine is an injection preparation or a nasal preparation.

7. The method for assisting losing weight in claim 6, wherein the injection preparation is a solution injection, a suspension injection, an emulsion injection, a lyophilized powder for injection or an aseptic powder for injection.

8. The method for assisting losing weight in claim 6, wherein the nasal preparation is nasal spray, a nasal drop, a nasal solution, a nasal pack or a nasal aerosol.

9. The method for assisting losing weight in claim 5, wherein the subject is a mammal.
